(19) United States
(12) Patent Application Publication
(10) Pub. No.: US 2024/0226139 A9
(43) Pub. Date: Jul. 11, 2024

(54) DOSING REGIMEN FOR NASAL CAVITY AND PHARYNX DECONGESTION

(71) Applicant: Graceful Caring Hands, Inc., Saline, MI (US)

(72) Inventor: Olukunle Jacob Adebiyi, Saline, MI (US)

(21) Appl. No.: 18/382,179

(22) Filed: Oct. 20, 2023

Related U.S. Application Data
(60) Provisional application No. 63/418,118, filed on Oct. 21, 2022.

(51) Int. Cl. A61K 33/00; A61K 9/00; A61K 9/08; A61K 31/09; A61K 31/495; A61P 11/02; A61P 11/04

(52) U.S. Cl. CPC A61K 33/00; A61K 9/0043; A61K 9/0056; A61K 9/0095; A61K 9/08; A61K 31/09; A61K 31/495; A61P 11/02; A61P 11/04

(57) ABSTRACT

A dosing regimen for administering a composition to a patient for treatment of congestion or irritation includes administering a first dose of the composition to the patient. The composition is formulated as an aqueous solution including between about 0.125 to about 1 milliequivalents of sodium bicarbonate per milliliter of the aqueous solution. The first dose includes between about 0.25 to about 1 milliliter of the composition. The dosing regimen also includes administering a second dose of the composition to the patient after administration of the first dose. The second dose includes between about 0.25 to about 1 milliliter of the composition.

### BACKGROUND OF THE INVENTION

### 1. Field of the Invention

The invention generally relates to a dosing regimen for nasal cavity and pharynx decongestion.

### 2. Description of the Related Art

As part of the respiratory tract, a healthy nose moisturizes and warms incoming air and filters out foreign materials. There are several defense mechanisms that protect the respiratory tract from infection by penetrating foreign bodies. For example, protection can be provided by mechanical barriers in the nasal cavity. Foreign particles that enter the nasal cavity or upper respiratory tract are trapped in mucus and carried back to the pharynx, where they are swallowed. For example, “congestion” can include a physiological response to an irritant that involves increased blood flow to tissues lining the nose. The increased blood flow causes these tissues to swell and physically block the nasal passages.

Intranasal administration is known to be an efficient, easy, and non-invasive method for delivery of agents and compounds into the bloodstream. Current medications and treatments for congestion provide only minimal symptom relief and have undesirable side effects. Over-the-counter medications containing antihistamines cause drowsiness and impair cognitive judgement. Some over-the-counter sprays focus on the reduction of blood flow to the nasal mucosa through the use of vasoconstrictors but provide little action with regard to other causes of nasal congestion.

Consequently, there is a need for a dosing regimen of an over-the-counter composition that provides safe, economical, and effective nasal and pharynx decongestion without the adverse side effects associated with existing treatments.

### SUMMARY OF THE INVENTION AND ADVANTAGES

A dosing regimen for administering a composition to a patient for treatment of congestion or irritation is provided. The dosing regimen includes administering a first dose of the composition to the patient. The composition is formulated as an aqueous solution including between about 0.125 to about 1 milliequivalents of sodium bicarbonate per milliliter of the aqueous solution. The first dose includes between about 0.25 to about 1 milliliter of the composition. The dosing regimen also includes administering a second dose of the composition to the patient after administration of the first dose. The second dose includes between about 0.25 to about 1 milliliter of the composition.

Accordingly, the dosing regimen provides safe, economical, and effective nasal and pharynx decongestion.

### DETAILED DESCRIPTION OF THE INVENTION

A dosing regimen for administering a composition to a patient for treatment of congestion or irritation is provided. The dosing regimen includes administering a first dose of the composition to the patient. The composition is formulated as an aqueous solution including between about 0.125 to about 1 milliequivalents of sodium bicarbonate per milliliter of the aqueous solution. The first dose includes between about 0.25 to about 1 milliliter of the composition. The dosing regimen also includes administering a second dose of the composition to the patient after administration of the first dose. The second dose includes between about 0.25 to about 1 milliliter of the composition. Accordingly, the dosing regimen provides safe, economical, and effective nasal and pharynx decongestion.

The composition is provided suitable for a patient in need of treatment of congestion. The composition includes about 0.125 to about 1 milliequivalent (mEq) of sodium bicarbonate per milliliter of the composition. As used herein, the term “includes” has the same meaning as “comprises.” Alternatively, the composition may consist of about 0.125 to about 1 milliequivalent of sodium bicarbonate per milliliter of the composition, or the composition may consist essentially of about 0.125 to about 1 milliequivalent of sodium bicarbonate per milliliter of the composition.

The composition disclosed herein may use an osmotic effect to aid in the decongestion of the nasal cavity and/or the pharynx of the patient, thus treating the patient of congestion. More specifically, the administration of the composition may result in osmotic forces driving water from the free spaces in the nasal tissue or pharynx tissue, for example the nasal mucosa tissue, and into the nasal tissues or the pharynx tissues, which shrinks the nasal mucosa or pharynx mucosa and provides extra fluid for moisturizing the nasal cavity and the pharynx and which increases movement of the mucus from the nasal cavity and the pharynx. As such, it is to be appreciated that the composition disclosed herein may increase the moisture inside a patient's nose, which is particularly advantageous during colds or allergic responses when the patient's nose is irritated and dry.

More specifically still, the composition may have an alkaline (i.e., basic) pH. The alkaline pH of the composition serves to aid in generation of the osmotic forces, thus lowering a viscosity of the mucus in a patient's nose. In some embodiments, the pH of the composition is between about 7 and about 8.5. If pH adjustment is needed, the amount of sodium bicarbonate in the composition and/or the amount of sodium bicarbonate administered to the patient may be adjusted.

The composition disclosed herein may be administered to any mammal. Preferably, the mammal is a human being. The term “therapeutically effective amount” refers to that amount which is sufficient to effect the desired change in the patient. The term “treatment” as used herein includes: (i) preventing a symptom or condition from occurring; (ii) inhibiting a symptom or condition, such as stopping its development; and/or (iii) relieving a symptom or condition.

The composition and methods disclosed herein are useful for loosening nasal and pharynx mucus, decongestion, and relieving nasal and pharynx irritation. Specifically, the composition and methods disclosed herein are used for the loosening of nose and pharynx mucus for easy expectoration and fast relief of congestion. The composition disclosed herein avoid the use of vasoconstrictors and aid in the decongesting and moisturizing of the nose by thinning and decreasing the viscosity of mucus. The composition disclosed herein also provides pharynx irritation relief to a patient by relieving a patient's itchy pharynx.

The composition disclosed herein may be administered over-the-counter. The therapeutically effective amount of the composition disclosed herein to administer to a patient vary depending on various factors, such as the method of administration, the age of the patient, the types of symptoms/conditions experienced by the patient, and the severity of symptoms/conditions experienced by the patient.

Although not required, the second dose may be administered after the first dose. If administered, the second dose is typically administered about 3 hours to about 6 hours after administration of the first dose. It is to be appreciated that the second dose may be administered about 3 hours to about 5 hours after administration of the first dose, may be administered about 3 hours to about 4 hours after administration of the first dose, may be administered about 4 hours to about 6 hours after administration of the first dose, may be administered about 5 hours to about 6 hours after administration of the first dose, may be administered about 3 hours after the first dose, may be administered about 4 hours after the first dose, may be administered about 5 hours after the first dose, or may be administered about 6 hours after the first dose.

Administration of the first dose in the dosing regimen may include directing the first dose to at least one body location chosen from a nasal cavity and a pharynx of the patient and retaining the first dose in the nasal cavity or the pharynx of the patient for at least two minutes. Moreover, administration of the second dose in the dosing regimen may include directing the second dose to at least one body location chosen from the nasal cavity and the pharynx of the patient and retaining the second dose in the nasal cavity or the pharynx of the patient for at least two minutes. It is to be appreciated that the at least one body location which the first dose and/or the second dose are directed to may include the nasal cavity, the pharynx, or both the nasal cavity and the pharynx. It is also to be appreciated that the first dose and/or the second dose may be retained for at least 3 minutes, at least 4 minutes, at least 5 minutes, between 2 minutes and 5 minutes, between 2 minutes and 4 minutes, between 2 minutes and 3 minutes, between 3 minutes and 5 minutes, between 3 minutes and 4 minutes, between 4 minutes and 5 minutes, about 2 minutes, about 3 minutes, about 4 minutes, or about 5 minutes. Retaining the first dose and/or the second dose in the nasal cavity or the pharynx of the patient permits time for osmosis in the nasal cavity and/or pharynx to occur and thus provide decongestion to the patient.

In one embodiment as described herein, the composition is formulated as the aqueous solution. In other embodiments, as will be described herein, the composition may be formulated as a lozenge or as a syrup. It is to be appreciated that the aqueous solution may have various viscosities and includes both water-based formulations as well as syrups. The lozenge may be formulated as an orally disintegrating tablet or a rapid dissolution tablet, among other possibilities.

Although not required, the composition may be formulated as an aqueous solution including about 0.125 milliequivalent of sodium bicarbonate per milliliter (0.05 mEq/ml) of the aqueous solution to about 1 milliequivalent of sodium bicarbonate per milliliter (1 mEq/ml) of the aqueous solution. Additionally, the composition may be formulated as the aqueous solution including between about 0.25 milliequivalents of sodium bicarbonate per milliliter (0.25 mEq/ml) of the aqueous solution to about 0.75 milliequivalents of sodium bicarbonate per milliliter (0.75 mEq/ml) of the aqueous solution, or the composition may be formulated as the aqueous solution including between about 0.25 milliequivalents of sodium bicarbonate per milliliter (0.25 mEq/ml) of the aqueous solution to about 0.5 milliequivalents of sodium bicarbonate per milliliter (0.5 mEq/ml) of the aqueous solution. Moreover, the composition may be formulated as the aqueous solution including about 0.75 milliequivalents of sodium bicarbonate per milliliter (0.75 mEq/ml) of the aqueous solution, about 0.5 milliequivalents of sodium bicarbonate per milliliter (0.5 mEq/ml) of the aqueous solution, or about 0.25 milliequivalents of sodium bicarbonate per milliliter (0.25 mEq/ml) of the aqueous solution. 1 milliequivalent is about 84 milligrams of sodium bicarbonate.

The dosing regimen may further include administering a third dose of the composition to the patient about 3 hours to about 6 hours after administration of the second dose. The third dose typically includes between about 0.25 milliliters to about 1 milliliter of the composition. Moreover, the third dose may be formulated as the aqueous solution and may include the same concentrations of sodium bicarbonate as described herein for the first dose and the second dose.

The composition, as formulated as the aqueous solution, may include a therapeutically effective amount of sodium bicarbonate for treatment of congestion without systemically affecting blood pH levels of the patient. As such, administration of the first dose and administration of the second dose in the dosing regimen may result in a therapeutically effective amount of sodium bicarbonate being administered for treatment of congestion without systemically affecting blood pH levels of the patient. It is to be appreciated that systemic effects on the blood pH levels of the patient include alkalosis of blood of the patient.

The aqueous solution may be formulated to be administered to the patient through a spray apparatus suitable for at least one of nasal cavity and pharynx administration. More specifically, administering the first dose may include metering a predetermined volume of the composition through the spray apparatus, and administering the second dose may include metering the predetermined volume of the composition through the spray apparatus. The predetermined volume may be between about 0.25 to about 0.5 milliliters. The spray apparatus may be configured to meter about 0.25 to about 1 milliliter per each spray, about 0.25 milliliters per spray, about 0.5 milliliters per each spray, about 0.75 milliliters per each spray, or about 1 milliliter per each spray. The spray apparatus may be various sizes including, but not limited to, 15 ml and 30 ml bottles.

Metering the predetermined volume of the composition through the spray apparatus to administer the first dose may include directing between about 0.25 to about 0.5 milliliters to the pharynx of the patient, and metering the predetermined volume of the composition through the spray apparatus to administer the second dose may include directing between about 0.25 to about 0.5 milliliters to the pharynx of the patient. In other words, the aqueous solution may be administered as an oral spray in the amount of 0.25 to about 0.5 milliliters of the composition metered per each spray to the pharynx of the patient. The aqueous solution may be held in bottles, with each bottle containing between about 30 milliliters to about 240 milliliters of the aqueous solution.

In another embodiment, metering the predetermined volume of the composition through the spray apparatus to administer the first dose includes directing about 0.25 milliliters through a first nostril to the nasal cavity of the patient, and directing about 0.25 milliliters through a second nostril to the nasal cavity of the patient. Moreover, in this same embodiment, metering the predetermined volume of the composition through the spray apparatus to administer the second dose includes directing about 0.25 milliliters through the first nostril to the nasal cavity of the patient, and directing about 0.25 milliliters through the second nostril to the nasal cavity of the patient. In other words, the aqueous solution may be administered as a nasal spray in the amount of about 0.25 milliliters of the composition metered per each spray, with each dose including one spray to the first nostril of the patient, and including one spray to the second nostril of the patient. The aqueous solution may be held in bottles, with each bottle containing between about 15 milliliters to about 60 milliliters of the aqueous solution.

Alternatively, the aqueous solution may be formulated to be administered through a liquid dropper having a liquid dropper bottle and an applicator such as a bulb syringe or squeeze dropper. The applicator, such as the bulb syringe or the squeeze dropper, may be calibrated to deliver 0.1 to 2 milliliters of the aqueous solution. The liquid dropper bottle may be various sizes including, but not limited to, 15 ml and 30 ml bottles. The liquid dropper may include an irrigation tube which may be inserted into the nasal cavity and/or the pharynx of the patient. Administering the first dose of the dosing regimen may include metering a predetermined volume of the composition through the liquid dropper, and administering the second dose of the composition may include metering the predetermined volume of the composition through the liquid dropper. The predetermined volume metered through the liquid dropper may be between about 0.25 to about 2 milliliters.

Moreover, metering the predetermined volume of the composition through the liquid dropper to administer the first dose may include directing between about 1 to about 2 milliliters of the composition to the pharynx of the patient, and metering the predetermined volume of the composition through the liquid dropper to administer the second dose may include directing between about 1 to about 2 milliliters of the composition to the pharynx of the patient. In the embodiments of the dosing regimen where the first dose and/or the second dose are administered via the liquid dropper to the pharynx of the patient, the aqueous solution may include about 0.25 to about 0.5 milliequivalents of sodium bicarbonate per milliliter of the aqueous solution.

Furthermore, metering the predetermined volume of the composition through the liquid dropper to administer the first dose may include directing between about 0.25 to about 1 milliliter through the first nostril to the nasal cavity of the patient, and directing about 0.25 to about 1 milliliter through the second nostril to the nasal cavity of the patient. Metering the predetermined volume of the composition through the liquid dropper to administer the second dose may include directing about 0.25 to about 1 milliliter through the first nostril to the nasal cavity of the patient, and directing about 0.25 to about 1 milliliter through the second nostril to the nasal cavity of the patient. In the embodiments of the dosing regimen where the liquid dropper is used to administer the composition through the first and second nostrils to the nasal cavity of the patient, the composition may include about 0.5 milliequivalents of sodium bicarbonate per milliliter of the aqueous solution.

In another route of administration of the dosing regimen, the first dose may be administered via nebulization to generate a mist including between about 0.125 to about 1 milliequivalents of sodium bicarbonate per milliliter of the mist, and the second dose may be administered via nebulization to generate the mist including about 0.125 to about 1 milliequivalents of sodium bicarbonate per milliliter of the mist. It is to be appreciated that nebulization may be used to generate the mist, which may also be referred to as an aerosol, of a droplet size which may be inhaled through the pharynx and into the lower respiratory tract of the patient. Nebulization may be accomplished through use of a nebulizer. The aqueous solution used for nebulization may be contained in 2 milliliter, 3 milliliter, 4 milliliter, or 5 milliliter ampules, among other possibilities.

The aqueous solution as formulated may be free of preservatives. The aqueous solution being free of preservatives may limit potential allergic reactions which the patient may have in responsive to a preservative. However, it is alternatively contemplated that the composition may include one or more preservatives and/or one or more additives, including but not limited to: anhydrous citric acid (having a maximum potency of about 0.12 mg/0.2 ml), particularly in embodiments where the composition is formulated as an aqueous solution; anhydrous citric acid (having a maximum potency of about 0.45 mg/1 ml), particularly in embodiments where the composition is formulated as an aqueous solution; anhydrous citric acid (having a maximum potency of about 0.45 mg/1 ml), particularly in embodiments where the composition is formulated as an aqueous solution; glycerin (having a maximum potency of about 25 mg/1 ml), particularly in embodiments where the composition is formulated as an aqueous solution; glycerin (having a maximum potency of about 22.3 mg/mL), particularly in embodiments where the composition is formulated as an aqueous solution; anhydrous trisodium citrate (having a maximum potency of about 2.54 mg/0.2 ml), particularly in embodiments where the composition is formulated as an aqueous solution; anhydrous trisodium citrate (having a maximum potency of about 48 mg/5 ml), particularly in embodiments where the composition is formulated as an effervescent powder; anhydrous trisodium citrate (having a maximum potency of 240 mg/day), particularly in embodiments where the composition is formulated as a lozenge; sorbitol (having a maximum potency of 25 mg/ml), particularly in embodiments where the composition is formulated as an aqueous solution; sorbitol (having a maximum potency of about 44.8 mg/ml), particularly in embodiments where the composition is formulated as an aqueous solution; benzyl alcohol (having a maximum potency of about 1.8 mg/0.2 ml), particularly in embodiments where the composition is formulated as an aqueous solution; benzyl alcohol (having a maximum potency of about 5 mg/l ml), particularly in embodiments where the composition is formulated as an aqueous solution; propylene glycol (having a maximum potency of about 49.7 mg/1 ml), particularly in embodiments where the composition is formulated as an aqueous solution; propylene glycol (having a maximum potency of about 50 mg/1 ml), particularly in embodiments where the composition is formulated as an aqueous solution; polysorbate 80 (having a maximum potency of about 0.05 mg), particularly in embodiments where the composition is formulated as an aqueous solution; polysorbate 80 (having a maximum potency of about 0.05 mg), particularly in embodiments where the composition is formulated as an aqueous solution; menthol (having a maximum potency of about 40.32 mg), particularly in embodiments where the composition is formulated as an aqueous solution; menthol (having a maximum potency of about 0.94 mg), particularly in embodiments where the composition is formulated as a lozenge; menthol (having a maximum potency of about 14 mg), particularly in embodiments where the composition is formulated as an orally disintegrating tablet; cellulose microcrystalline/carboxymethylcellulose sodium (having a maximum potency of about 60 mg), particularly in embodiments where the composition is formulated as an aqueous solution; and any combinations thereof.

The aqueous solution as formulated may be sterile. As such, the aqueous solution may be prepared aseptically. Thus, the aqueous solution may limit potential infection of the patient. The composition may include other medicinal fragrances and flavoring agents including, but not limited to, pineapple flavor oil, vanilla bean flavor oil, oregano and cucumber melon flavor oil to aid in increasing the palatability of the composition.

The aqueous solution may include about 0.25 to about 1 milliequivalent of sodium bicarbonate per milliliter of the aqueous solution and may include about 0.25 to about 0.75 milliequivalent of sodium bicarbonate per milliliter of the aqueous solution. In one embodiment, the aqueous solution includes about 0.25 milliequivalent of sodium bicarbonate per milliliter of the aqueous solution. In another embodiment, the aqueous solution includes about 0.5 milliequivalent of sodium bicarbonate per milliliter of the aqueous solution. In yet another embodiment, the aqueous solution includes about 1 milliequivalent of sodium bicarbonate per milliliter of the aqueous solution.

As described herein, the composition may be formulated as an effervescent powder. The effervescent powder may include about 0.05 to about 3 milliequivalent of sodium bicarbonate. Prior to administering sterile solution of the sodium bicarbonate in water, sodium bicarbonate may be found in a white, crystalline powder. The powder should be stored in dry (not moist) air to avoid degradation. The sodium bicarbonate solution should be stored at about 15-40 degrees C. in a tightly closed container. Freezing of the sodium bicarbonate solution should be avoided.

The composition disclosed herein may be provided in a kit comprising: (i) a 0.5 mEq/ml preservative-free sterile solution in 15 ml and/or 30 ml bottles; and (ii) a sachet of 1 teaspoonful pure sodium bicarbonate powder to be added to 120 ml or 60 ml of sterile water to make the 0.5 mEq/ml solution or 1 mEq/ml respectively and a ½ teaspoonful sachet to be added to 60 ml or 120 ml sterile water to make a 0.5 mEq or 0.25 mEq sodium bicarbonate solution, respectively.

Alternatively, as also described herein, the composition may be formulated as a lozenge. The lozenge may include a solid base and about 0.25 to about 1 milliequivalent of sodium bicarbonate per each lozenge. The solid base may include honey, although other formulations of solid bases are contemplated. The honey may be pure (i.e., free of impurities) and may be any type of honey. More specifically, although not required, the lozenge may include about 0.05 to about 1 milliequivalent of sodium bicarbonate per milliliter of the honey. Although the honey may be crystallized in the solid base, it is to be appreciated that the honey may be measured in milliliters when in a liquid form. It is also to be appreciated that the lozenge may include additional components, such as sweeteners, and may be of any size.

The lozenge may also further include guaifenesin. Guaifenesin is an expectorant that typically loosens and thins mucus and bronchial secretions. Irritation of gastric vagal receptors responsible for stimulation of respiratory tract fluid result in an increase of volume and decrease in viscosity of respiratory tract secretions. Cough reflex sensitivity may therefore be inhibited, for example by guaifenesin, in a patient experiencing upper respiratory tract infection in whom cough receptors are transiently hypersensitive. If included, the lozenge typically includes about 25 to about 100 milligrams of guaifenesin. The lozenge may also include about 0.25 to about 1 milliequivalent of sodium bicarbonate. It is to be appreciated that the lozenge may include a ratio of about 1 milligram of guaifenesin to about 0.01 milliequivalent of sodium bicarbonate. More specifically, the lozenge may further include a ratio of about 1 milligram of guaifenesin to about 0.01 milliequivalent of sodium bicarbonate to about 0.05 to about 0.2 ml of the honey.

In another embodiment of the dosing regimen, the dosing regimen includes administering a first dose of the composition to the patient, with the composition formulated as one or more lozenges each including the solid base, between about 0.5 to about 1 milliequivalents of sodium bicarbonate, and between about 50 to about 100 milligrams of guaifenesin. For this embodiment, the dosing regimen may also include administering a second dose of the composition to the patient after administration of the first dose, with the composition also being formulated as one or more lozenges in the second dose.

In one embodiment, the lozenge includes about 25 mg of guaifenesin and about 0.25 milliequivalent of sodium bicarbonate, optionally per about 5 milliliters of the honey, which optionally may be administered to a child of 4-6 years of age in about one lozenge per dose every about 4 hours to about 6 hours. In another embodiment, the lozenge includes about 50 mg of guaifenesin and about 0.5 milliequivalent of sodium bicarbonate, optionally per about 5 milliliters of the honey, which optionally may be administered to a child of 6-12 years of age in about one lozenge per dose every about 4 hours to about 6 hours. In yet another embodiment, the lozenge includes about 100 mg of guaifenesin and about 1 milliequivalent of sodium bicarbonate, optionally per about 5 milliliters of the honey, which optionally may be administered to an adolescent of 12-16 years of age in about 1 lozenge per dose every about 4 to about 6 hours. In yet another embodiment, the lozenge includes about 100 mg of guaifenesin and about 1 milliequivalent of sodium bicarbonate, optionally per about 5 milliliters of honey, which optionally may be administered to an adolescent over 16 years of age or to an adult in about 1 or about 2 lozenges per dose every about 4 hours to about 6 hours. In the embodiments described above, it is further to be appreciated that the lozenges described herein may be administered to children, adolescents, and adults as needed for cough, throat irritation, nasal congestion, and/or throat congestion as determined by the patient and/or their medical provider in accordance with the symptomatic presentation of the patient. Although not required, each lozenge may weigh between 1.25 grams to about 4.5 grams.

In another embodiment, the dosing regimen for administering the composition to a patient for treatment of congestion or irritation includes administering a first dose of the composition to the patient, with the first dose formulated as a syrup comprising a liquid base, between about 0.25 to about 0.5 milliequivalents of sodium bicarbonate per milliliter of the liquid base, and between about 2.5 to about 20 milligrams of guaifenesin per milliliter of the liquid base. The dosing regimen may also include administering a second dose of the composition to the patient after administration of the first dose, with the second dose also formulated as the syrup comprising the liquid base, between about 0.25 to about 0.5 milliequivalents of sodium bicarbonate per milliliter of the liquid base, and between about 2.5 to about 20 milligrams of guaifenesin per milliliter of the liquid base.

The syrup may further include cetirizine hydrochloride. In a non-limiting example, the syrup may further include between about 0.25 to about 2 milligrams of cetirizine hydrochloride per milliliter of the liquid base. Cetirizine hydrochloride is a second-generation antihistamine and acts as a highly selective antagonist of the histamine H1 receptor in the patient. The syrup may be held in a bottle, with each bottle including between about 30 milliliters and about 120 milliliters of the syrup.

The aqueous solution may also include guaifenesin. The aqueous solution may include about 25 to about 100 milligrams of guaifenesin. The aqueous solution may also include about 0.25 to about 1 milliequivalent of sodium bicarbonate per milliliter of the composition. It is to be appreciated that the aqueous solution may include a ratio of about 1 milligram of guaifenesin to about 0.01 milliequivalent of sodium bicarbonate. More specifically, the aqueous solution may further include a ratio of about 1 milligram of guaifenesin to about 0.01 milliequivalent of sodium bicarbonate to about 0.05 to about 0.2 ml of the honey. The aqueous solution including guaifenesin may be held in bottles, with each bottle including between about 30 milliliters and about 120 milliliters of the aqueous solution.

In one embodiment, the aqueous solution includes about 25 mg of guaifenesin and about 0.25 milliequivalent of sodium bicarbonate, optionally per about 2.5 milliliters of the honey, which optionally may be administered to a child of 4-6 years of age in about one dose of about 2.5 milliliters to about 5 milliliters every about 4 hours to about 6 hours. In another embodiment, the aqueous solution includes about 50 mg of guaifenesin and about 0.5 milliequivalent of sodium bicarbonate, optionally per about 2.5 milliliters of the honey, which optionally may be administered to a child of 6-12 years of age in about one dose of about 2.5 milliliters to about 5 milliliters every about 4 hours to about 6 hours. In yet another embodiment, the aqueous solution includes about 100 mg of guaifenesin and about 1 milliequivalent of sodium bicarbonate, optionally per about 2.5 milliliters of the honey, which optionally may be administered to an adolescent of 12-16 years of age in about dose of about 2.5 milliliters to about 5 milliliters every about 4 to about 6 hours. In yet another embodiment, the aqueous solution includes about 100 mg of guaifenesin and about 1 milliequivalent of sodium bicarbonate, optionally per about 2.5 milliliters of honey, which optionally may be administered to an adolescent over 16 years of age or to an adult in about 1 dose of about 2.5 milliliters to about 5 milliliters every about 4 hours to about 6 hours. In the embodiments described above, it is further to be appreciated that the aqueous solutions described herein may be administered to children, adolescents, and adults as needed for cough, throat irritation, nasal congestion, and/or throat congestion as determined by the patient and/or their medical provider in accordance with the symptomatic presentation of the patient.

The composition disclosed herein may be standardized. In other words, the composition disclosed herein may comprise a predetermined amount of sodium bicarbonate, a predetermined amount of guaifenesin, and/or a predetermined amount of honey. Through standardization of doses, the patient is able to more accurately treat congestion and associated symptoms while limiting the risk of adverse reactions (e.g. systemic reactions, alkalosis).

A method of administering the composition to a patient in need of treatment of congestion is also provided. The method includes the step of administering to the patient in need of treatment of congestion the composition including about 0.05 to about 1 milliequivalent of sodium bicarbonate per milliliter of aqueous solution in at least one chosen from a throat of the patient, a nasal passage of the patient, and a pharynx of the patient. The composition may be retained in the throat, the nasal passage, and/or the pharynx of the patient for at least two minutes, for at least three minutes, for at least four minutes, and/or for at least five minutes. The onset of action in a patient may occur rapidly after administration of the composition. For example, the onset may occur within about three minutes to about five minutes of application of the composition. The effect of the composition may be sustained in the patient and may last several hours without having the patient experience any rebound effect.

About 0.25 to about 8 milliliters may be administered by the method, particularly in a single dose. More specifically, about 1 milliliter to about 4 milliliters may be administered by the method, particularly in a single dose, and about 1 milliliter to about 2 milliliters may be administered, particularly in a single dose. About 0.25 to about 3 milliequivalent of sodium bicarbonate per milliliter of the composition may be administered by the method, particularly in a single dose. More specifically, about 1 to about 2 milliequivalent of sodium bicarbonate per milliliter of the composition may be administered, particularly in a single dose.

The step of administering the composition may include administering the composition to a throat of the patient. Alternatively or additionally, the step of administering the composition may include administering the composition to a nasal passage of the patient. Alternatively or additionally, the step of administering the composition may include administering the composition to a pharynx of the patient. The step of administering the composition may include inserting an irrigation tube into at least one chosen from the throat of the patient, a nasal passage of the patient, and a pharynx of the patient and directing the composition through the irrigation tube. In other embodiments, the step of administering the composition may include spraying the composition using the spray apparatus. In yet other embodiments, the step of administering the composition may include dropping the composition using the liquid dropper.

The patient may recline their head before the composition is administered. In a non-limiting example, the patient may recline their head before, after, or concurrently with the irrigation tube being inserted, and the composition may be administered through the irrigation tube while the patient's head is reclined. In another non-limiting example, the patient may recline their head and the composition may be sprayed into one of the patient's nasal passages, throat, and pharynx using the spray apparatus while the patient's head is reclined. In yet another non-limiting example, the patient may recline their head and the composition may be dropped into one of the patient's nasal passages, throat, and pharynx using the liquid dropper. It is to be appreciated that the patient may inhale all or some of the composition, particularly when the composition is administered while the patient's head is reclined, which allows maximum retention of the composition and results in maximum therapeutic effect.

Another embodiment of the method is provided. The method of administering the composition to a patient in need of treatment of congestion includes the steps of mixing the composition formulated as an effervescent powder including about 0.05 to about 3 milliequivalent of sodium bicarbonate with about 1 to about 5 milliliters of water to form an aqueous solution including about 0.05 to about 1 milliequivalent of sodium bicarbonate per milliliter of the aqueous solution, and administering to the patient in need of treatment of congestion the composition formulated as the aqueous solution including about 0.05 to about 1 milliequivalent of sodium bicarbonate per milliliter of the aqueous solution in at least one chosen from a throat of the patient, a nasal passage of the patient, and a pharynx of the patient. The step of mixing may be further defined as mixing the composition formulated as the effervescent powder with about 2 to about 4 milliliters of water. More specifically, the step of mixing may be further defined as mixing the composition formulated as the effervescent powder with about 3 milliliters of water.

It will, of course, be understood that, although particular examples have been described and are described further below, the claimed subject matter is not limited in scope to a particular example or limitation. Likewise, an example may be implemented in any combination of compositions of matter, methods, or products made by a process, for example. The following examples are merely representative of methods of practicing one or more embodiments of the present disclosure and should not be read as limiting the scope of the disclosure.

Example 1: Dosing for Treating Nasal Congestion in Adults: For light or moderate nasal congestion, 0.25 ml-0.5 ml of 0.5 mEq/ml of sodium bicarbonate solution in preservative-free sterile water for injection is administered to each of a patient's nostril 3-4 times every day or as needed for treating the congestion. For severe nasal congestion, 0.5 ml-1 ml of 0.5 mEq/ml or 0.5 ml of 1 mEq/ml of sodium bicarbonate solution in preservative-free sterile water for injection is administered to each of a patient's nostril 3-4 times every day or as needed for treating the congestion.

Example 2: Dosing for Treating Throat Irritation and Congestion in Adults: Administer 1 ml of 0.5 mEq or 2 ml (1 mEq) of sodium bicarbonate solution to a patient orally 3-4 times every day or as needed for treating throat irritation and/or congestion. Administer 1-2 oral lozenges to a patient 3-4 times every day or as needed for treating throat irritation and/or congestion.

Example 3: Dosing for Treating Nasal Congestion in Adults and Children Over 12 Years Old: Using a graduated one millimeter (1 ml) pipette syringe, instill 0.25 ml (5 drops) into each nostril of a patient 2-3 times a day or as needed for nasal and throat congestion relief. One drop in the syringe is 0.05 ml and 10 drops in the syringe is 0.5 ml. Alternatively, the dose may be drawn up in the syringe and given orally.

Example 4: Dosing for Treating Nasal Congestion in Children Between 2/5 Years Old and 12 Years Old: Using a graduated one millimeter (1 ml) pipette syringe, instill 0.125 ml to 0.25 ml into each nostril of a patient 2-3 times a day or as needed for nasal and throat congestion relief. One drop in the syringe is 0.05 ml and 10 drops in the syringe is 0.5 ml. Alternatively, the dose may be drawn up in the syringe and given orally.

The invention has been described in an illustrative manner, and it is to be understood that the terminology which has been used is intended to be in the nature of words of description rather than of limitation. Many modifications and variations of the present invention are possible in light of the above teachings, and the invention may be practiced otherwise than as specifically described.

What is claimed is:

1. A dosing regimen for administering a composition to a patient for treatment of congestion or irritation, the dosing regimen comprising:
administering a first dose of the composition to the patient, with the composition formulated as an aqueous solution comprising between about 0.125 to about 1 milliequivalents of sodium bicarbonate per milliliter of the aqueous solution, and with the first dose including between about 0.25 to about 1 milliliter of the composition; and
administering a second dose of the composition to the patient after administration of the first dose, with the second dose including between about 0.25 to about 1 milliliter of the composition.

2. The dosing regimen as set forth in claim 1, wherein the second dose is administered about 3 hours to about 6 hours after the administration of the first dose.

3. The dosing regimen as set forth in claim 1, wherein administration of the first dose includes directing the first dose to at least one body location chosen from a nasal cavity and a pharynx of the patient and retaining the first dose in the nasal cavity or pharynx of the patient for at least two minutes, and wherein administration of the second dose includes directing the second dose to at least one body location chosen from the nasal cavity and the pharynx of the patient and retaining the second dose in the nasal cavity or pharynx of the patient for at least two minutes.

4. The dosing regimen as set forth in claim 1, wherein the aqueous solution comprises between about 0.25 and about 0.75 milliequivalents of sodium bicarbonate per milliliter of the aqueous solution.

5. The dosing regimen as set forth in claim 1, wherein the aqueous solution comprises about 0.5 milliequivalents of sodium bicarbonate per milliliter of the aqueous solution.

6. The dosing regimen as set forth in claim 1, wherein the aqueous solution comprises about 0.25 milliequivalents of sodium bicarbonate per milliliter of the aqueous solution.

7. The dosing regimen as set forth in claim 1 further comprising administering a third dose of the composition to the patient about 3 hours to about 6 hours after administration of the second dose, with the third dose including between about 0.25 to about 1 milliliter of the composition.

8. The dosing regimen as set forth in claim 1, wherein administration of the first dose and administration of the second dose results in a therapeutically effective amount of sodium bicarbonate being administered for treatment of congestion without systemically affecting blood pH levels of the patient.

9. The dosing regimen as set forth in claim 1, wherein administering the first dose includes metering a predetermined volume of the composition through a spray apparatus, wherein administering the second dose includes metering the predetermined volume of the composition through the spray apparatus, and wherein the predetermined volume is between about 0.25 to about 0.5 milliliters.

10. The dosing regimen as set forth in claim 9, wherein metering the predetermined volume of the composition through the spray apparatus to administer the first dose includes directing between about 0.25 to about 0.5 milliliters to a pharynx of the patient, and wherein metering the predetermined volume of the composition through the spray apparatus to administer the second dose includes directing between about 0.25 to about 0.5 milliliters to the pharynx of the patient.

11. The dosing regimen as set forth in claim 9, wherein metering the predetermined volume of the composition through the spray apparatus to administer the first dose includes directing about 0.25 milliliters through a first nostril to a nasal cavity of the patient and directing about 0.25 milliliters through a second nostril to the nasal cavity of the patient, and wherein metering the predetermined volume of the composition through the spray apparatus to administer the second dose includes directing about 0.25 milliliters through the first nostril to the nasal cavity of the patient and directing about 0.25 milliliters through the second nostril to the nasal cavity of the patient.

12. The dosing regimen as set forth in claim 11, wherein the aqueous solution comprises between about 0.25 to about 0.5 milliequivalents of sodium bicarbonate per milliliter of the aqueous solution.

13. The dosing regimen as set forth in claim 1, wherein administering the first dose includes metering a predetermined volume of the composition through a liquid dropper, wherein administering the second dose includes metering the predetermined volume of the composition through the liquid dropper, and wherein the predetermined volume is between about 0.25 to about 2 milliliters.

14. The dosing regimen as set forth in claim 13, wherein metering the predetermined volume of the composition through the liquid dropper to administer the first dose includes directing between about 1 to about 2 milliliters of the composition to a pharynx of the patient, wherein metering the predetermined volume of the composition through the liquid dropper to administer the second dose includes directing between about 1 to about 2 milliliters of the composition to a pharynx of the patient, and wherein the aqueous solution comprises about 0.25 to about 0.5 milliequivalents of sodium bicarbonate per milliliter of the aqueous solution.

15. The dosing regimen of claim 13, wherein metering the predetermined volume of the composition through the liquid dropper to administer the first dose includes directing between about 0.25 to about 1 milliliters through a first nostril to a nasal cavity of the patient and directing about 0.25 to about 1 milliliters through a second nostril to the nasal cavity of the patient, wherein metering the predetermined volume of the composition through the liquid dropper to administer the second dose includes directing about 0.25 to about 1 milliliters through the first nostril to the nasal cavity of the patient and directing about 0.25 to about 1 milliliters through the second nostril to the nasal cavity of the patient, wherein the aqueous solution comprises about 0.5 milliequivalents of sodium bicarbonate per milliliter of the aqueous solution.

16. The dosing regimen as set forth in claim 1, wherein the first dose is administered via nebulization to generate a mist including between about 0.125 to about 1 milliequivalents of sodium bicarbonate per milliliter of the mist, and wherein the second dose is administered via nebulization to generate a mist including about 0.125 to about 1 milliequivalents of sodium bicarbonate per milliliter of the mist.

17. A dosing regimen for administering a composition to a patient for treatment of congestion or irritation, the dosing regimen comprising:
administering a first dose of the composition to the patient, with the composition formulated as one or more lozenges each comprising a solid base, between about 0.5 to about 1 milliequivalents of sodium bicarbonate, and between about 50 to about 100 milligrams of guaifenesin; and
administering a second dose of the composition to the patient after administration of the first dose.

18. A dosing regimen for administering a composition to a patient for treatment of congestion or irritation, the dosing regimen comprising:
administering a first dose of the composition to the patient, with the first dose formulated as a syrup comprising a liquid base, between about 0.25 to about 0.5 milliequivalents of sodium bicarbonate per milliliter of the liquid base, and between about 2.5 to about 20 milligrams of guaifenesin per milliliter of the liquid base; and
administering a second dose of the composition to the patient after administration of the first dose.

19. The dosing regimen as set forth in claim 18, wherein the syrup further comprises cetirizine hydrochloride.

20. The dosing regimen as set forth in claim 19, wherein the syrup further comprises between about 0.25 to about 2 milligrams of cetirizine hydrochloride per milliliter of the liquid base.
